(19) United States
(12) Plant Patent Application Publication
(10) Pub. No.: US 2022/0418177 P1
(43) Pub. Date: Dec. 29, 2022

(54) APPLE TREE NAMED 'MD-TAP2'

(50) ;

(71) Applicant: University of Maryland, College Park, College Park, MD (US)

(72) Inventors: Christopher S. Walsh, University Park, MD (US); Julia M. Harshman, Ventura, CA (US); Kathleen W. Hunt, Seabrook, MD (US)

(21) Appl. No.: 17/663,325

(22) Filed: May 13, 2022

Related U.S. Application Data
(60) Provisional application No. 63/201,849, filed on May 14, 2021.

(51) Int. Cl. A01H 6/74

(52) U.S. Cl. PLT/161

(57) ABSTRACT

A new and distinctive variety of Malus domestica Borkh, apple tree named ‘MD-TAP2’ is distinguished by its attractive tree architecture, reduced need for hand pruning and training, observed tolerance to high temperatures during the growing season, fireblight (Erwinia amylovora) and fruit which is harvested two weeks earlier than its seed parent.

### CROSS REFERENCE TO RELATED APPLICATION

This application claims priority under 35 U.S.C. § 119 to provisional application Ser. No. 63/201,849, entitled Apple Tree Named ‘F333’, filed May 14, 2021, herein incorporated by reference in its entirety.

Latin name: Malus domestica.

Varietal denomination: ‘MD-TAP2’ (formerly F333)

### BACKGROUND

The invention refers to a new plant variety of apple tree (Malus domestica) named ‘MD-TAP2’. The new variety is distinguished by its attractive tree architecture, reduced need for hand pruning and training, and fruit which is harvested earlier than its seed parent. ‘MD-TAP2’ originated as a single seedling from an open pollination of the variety ‘Fuji’ (Unpatented). The seed-parent ‘Fuji’ tree was open pollinated, grown in an isolated block of seedlings, which in turn had been derived from a 1991 open pollination of the columnar apple tree, ‘McIntosh Wijcik’ (U.S. Plant Pat. No. 4,382). The original ‘MD-TAP2’ seedling was produced in 2002, germinated, grown in the University of Maryland greenhouse, and then transplanted at the Western Maryland Research and Education Center, Keedysville, Washington County, Md. in 2003. This original tree was observed to produce large, good-quality fruit annually during subsequent seasons. In 2015 budwood was taken from this original ‘MD-TAP2’ tree and bench-grafted onto clonal, non-dwarfing rootstocks of ‘M.111’. Grafted trees were planted at the University of Maryland farm in Keedysville. They were observed from 2016 until 2021. During that time, the leaves, flowers, fruit, shoots and trunk were found to be stable and essentially similar to those on the original seedling tree.

### BRIEF SUMMARY

The ‘MD-TAP2’ variety exhibits an exceptional apple tree architecture. With its mesotonic growth habit, wide-angle branches, shoot di-morphy leading to a spur-type habit, the tree canopy is open. Most fruiting shoots (spurs) receive full sunlight. Shoots are not rigid and arch downward with a typical crop load. Buds from the original seedling tree were vegetatively propagated by grafting on ‘M.111’ rootstocks. These were precocious; they began to flower and fruit in their third growing season. Despite limited training and pruning and grafting onto a non-dwarfing root stock, trees were manageable after six growing seasons. The fruit size of ‘MD-TAP2’ is large and similar to that of its seed parent ‘Fuji’, but matures earlier than that variety. It is a mid-to-late-September apple when grown in Keedysville, Md. In addition to maturing two weeks earlier than ‘Fuji’, the tree exhibits some field tolerance to fireblight, and the fruit does not appear to be susceptible to preharvest water core. These two problems occur when ‘Fuji’ fruit are produced in Maryland and other locations.

### BRIEF DESCRIPTION OF THE DRAWINGS

This new apple variety, ‘MD-TAP2’, is illustrated by the accompanying photographic drawings of the plant growing at the research farm in Washington County, Md. These drawings show the tree using color photography. Colors shown are approximate as they can depend on horticultural practices, weather conditions, and management strategies. Consequently the color characteristics of this new variety should be determined from the observations described herein rather than these drawings.

FIG. 1 is a color photograph taken on Apr. 13, 2022 of the original unpruned seedling apple tree ‘MD-TAP2’ during dormancy, showing its natural tree architecture.

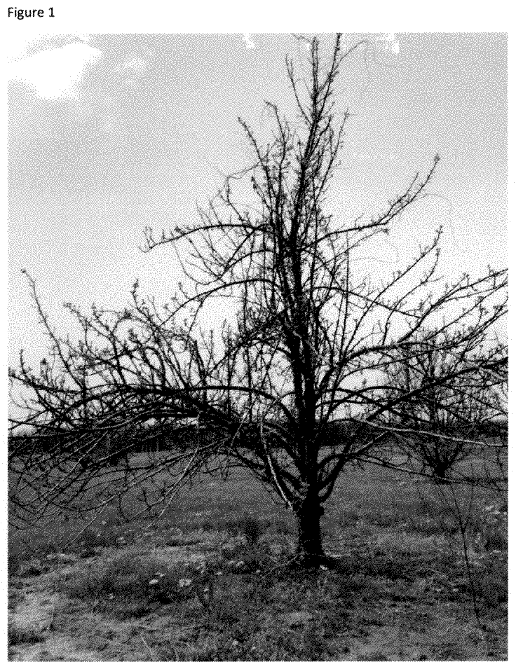

FIG. 2 is a color photograph taken on Oct. 2, 2017 of leaves and fruit on a single upright shoot of ‘MD-TAP2’ apple tree.

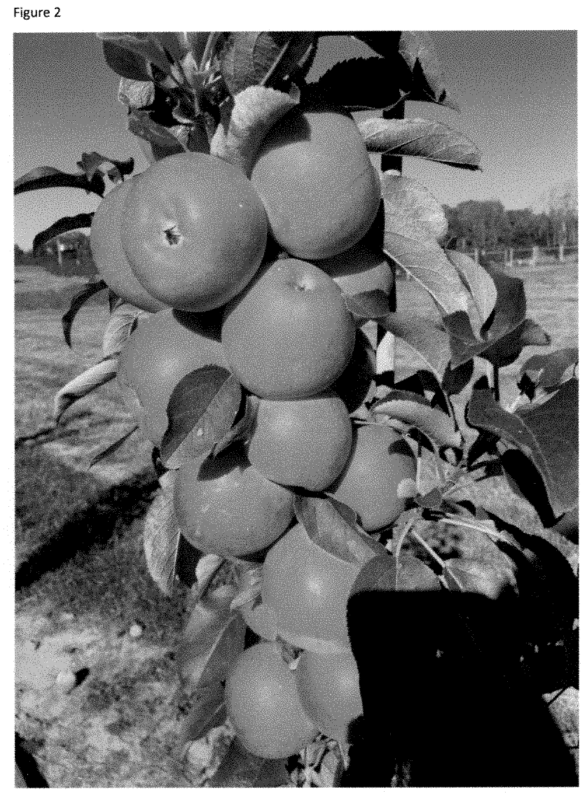

FIG. 3 is a color photograph taken on Sep. 30, 2019 of ‘MD-TAP2’ apple tree with a step ladder showing an upright branch being bent down with the weight of ripening fruit.

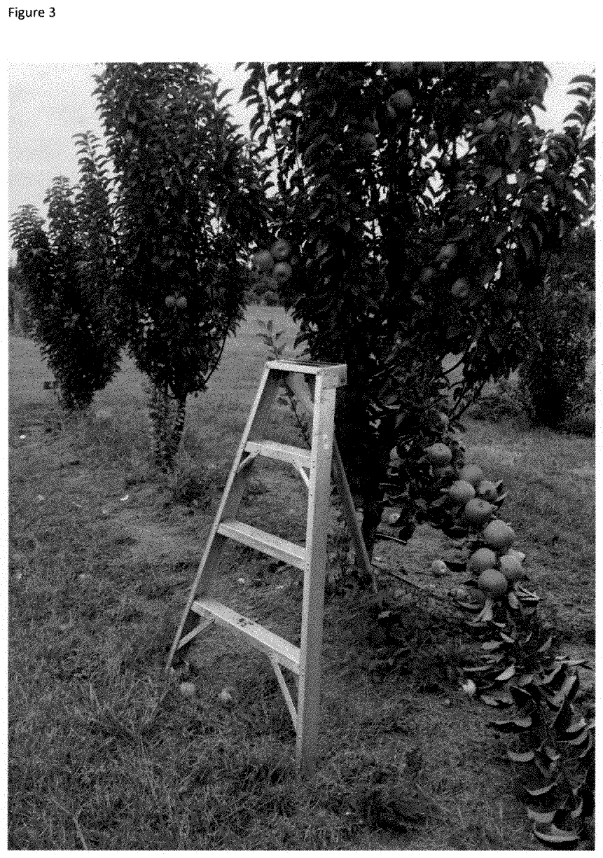

FIG. 4 is a color photograph taken on Sep. 30, 2019 of ‘MD-TAP2’ apple fruit harvested showing typical fruit over color, ground color, stems and calyces.

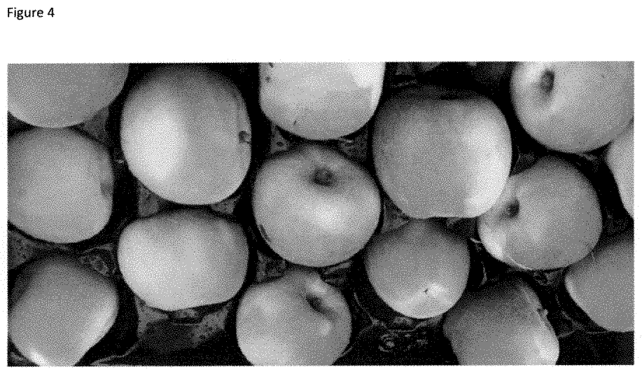

FIG. 5 is a color photograph taken on Apr. 14, 2021 of ‘MD-TAP2’ apple bloom showing fully opened flowers and unopened flower buds.

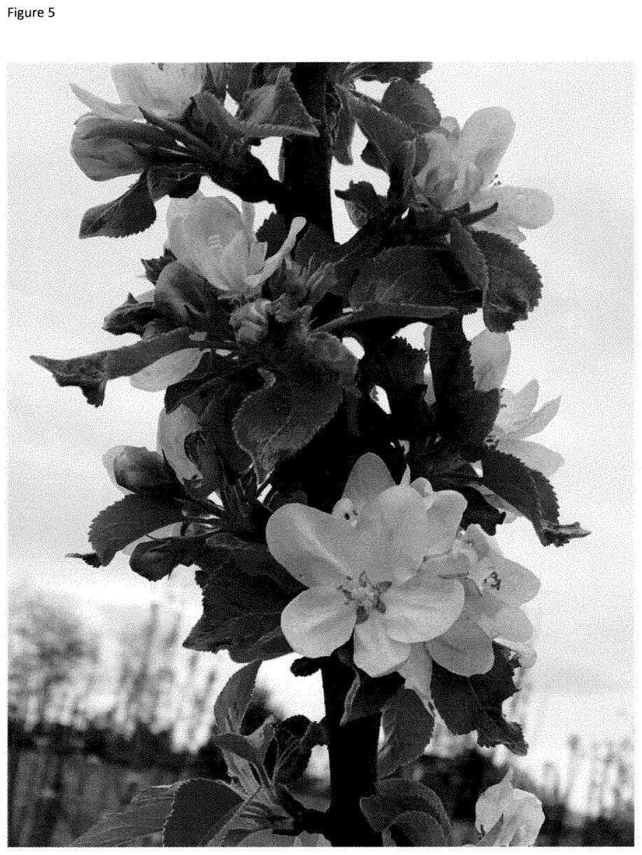

### DETAILED BOTANICAL DESCRIPTION

The following detailed description of ‘MD-TAP2’ trees budded onto ‘M.111’ rootstock grown at Keedysville, Washington County, Md. Measurements of the fruit were taken in 2020, and the measurements of the flowers, leaves and wood were taken in 2021. All color references are taken from The Royal Horticultural Society (R.H.S.) Colour Chart.
Scientific name: Malus x domestica Borkh.
Parentage:
   Seed parent.—‘Fuji’.
   Pollen parent.—Unnamed seedling apple tree derived from a 1991 ‘McIntosh Wijcik’ x ‘Gala’ cross created in Maryland.
Tree:
   Vigor.—Moderate.
   Plant hardiness zone.—Growth of plants has been observed in USDA Zone 6b.
   Dormant flower bud cold tolerance.—At least −5° F. (−17.8° C.).
   Overall shape.—Cone.
   Height.—13.4 feet (4.08 cm).
   Width.—12.5 feet (3.82 cm).
Trunk and branches:
   Trunk bark color.—Greyed-brown (RHS 199A).
   Primary branches.—Grey (RHS 201A).
   Branch angle.—Ranges between 30 and 90° from the trunk.
   Lenticels.—4.6 per cm.², Yellow-white (RHS 158A).
   Terminal shoots.—51 cm. in length; mid-shoot diameter 0.9 cm.
   New growth bark.—Brown (RHS 200A).
Leaves:
   Texture.—Upper surface smooth; lower surface slightly velvety.
   Length.—11.8 cm.
   Width.—5.1 cm.
   Apex.—Acute.
   Venation.—Pinnate.
   Margin.—Serrated with about seven teeth per cm.
   Petioles.—Between 2.9 and 3.5 cm. long. Color is Greyed-red (RHS 181A).
   Stipules.—Two with an average length of 8 mm.
Leaf color:
   Upper leaf surface.—Green (RHS N137A).
   Lower leaf surface.—Yellow-green (RHS 148B).
   Vein.—Yellow-green (RHS 145C).
Flowers:
   Size.—7.8 cm. diameter.
   Petals.—5 ovate petals per flower; Petal length 2.5 cm. and width 1.8 cm.
   Pistil.—8.8 mm. tall; divides into 5 stigmatic services, 3.6 mm. above the point of attachment.
   Stigma color.—Yellow-green (RHS 145A).
   Stamens.—Typically 18; anther color, Greyed-yellow (RHS 162B); filament Yellow-green (RHS 145D).
   Pollen.—Brilliant yellow (RHS 11A); abundance medium.
   Flower sepals.—Length 5 mm.; Reflexed.
   Flower calyx shape.—Spurred; Meeting at the base.
   Color.—Green (RHS 142C).
   Pedicel length.—16.1 mm.
   Bloom season.—Mid-season.
Fruit:
   Size.—Medium, averaging 7.3 cm. diameter and 178 gram weight.
   Shape.—Globose.
   Color.—Overcolor is an Orange-red (RHS N34A) blush; ground color is Yellow-green (RHS 151B).
   General color effect.—Brightly colored Red (RHS 34B) over Yellow-green background (RHS 151B) and a yellow-green stem cavity.
   Skin.—Smooth and waxy.
   Stem.—Variable length, ranging from 11 mm. to 18 mm. and 3.0 mm. diameter at the mid-point.
   Cavity.—Flaring and smooth. Sometimes a minimal amount of russet.
   Basin.—Diameter ranges from 26 mm. to 37 mm. depending on fruit size.
   Calyx.—Calyx basin smooth; 5 sepals present.
   Calyx tube—Cone-shaped, Sepals persistent with closed upright tips.
   Lenticels.—25 per cm.²; Greyed-yellow (RHS 162C).
   Russet—Minimal to none.
Fruit properties:
   Flesh color.—Yellow (RHS 10D).
   Juice.—Above average.
   Firmness.—Firm; 16.0 lb. at harvest.
   Texture.—Crisp and smooth.
   Flavor.—Mild and aromatic.
   Soluble solids.—11.9° Brix.
   Aroma.—Pleasant.
   Eating quality.—Very good.
   Keeping quality.—Best before 4-5 months storage.
   Harvest date.—September 20-30 in Washington County, Md.
   Productivity of the fruit.—Research planting yielded about 2 bushels per tree at a spacing of 330 trees per acre; at 42 lbs. per bushel, the estimated yield would be 27,720 lbs. per acre.
   Core.—Median position; 3 cm. in diameter.
   Core lines.—Basal; Clasping.
   Carpellary area.—Visible.
   Depth of calyx tube.—10 mm.
   Seed cells.—Generally 1-2 seeds per cell. Closed; 5 in number.
   Cell walls.—Thick and firm.
Seeds:
   Number.—7 to 10.
   Length.—11 mm.
   Breadth.—5 mm.
   Form.—Pyriform, resembling a teardrop.
   Color.—Greyed-orange (RHS 166A).
   Plant/fruit disease and pest resistance/susceptibility—In addition to tree architecture which reduces pruning and training labor, ‘MD-TAP2’ trees have shown acceptable field tolerance to fireblight (Erwinia amylovora) when grown in the Washington County, Md. research orchard. Due to heavy fruit set, trees will benefit from standard crop-load management practices.
Pollination: Diploid apples with an early to mid-season bloom date. Compatibility with ‘Fuji,’ its seed parent, has not been tested. Due to its early bloom and parentage, some flowers may be susceptible to late spring freezes, similar to ‘Delicious’ and ‘Fuji.’
Use: For dessert.

### REFERENCES

Royal Horticultural Society. 2007. R.H.S. Colour Chart (5^{th} Edition). London SW1P 2PE.

What is claimed is:

1. A new and distinct apple variety named ‘MD-TAP2’ as shown and described herein.
